(19) United States
(12) Patent Application Publication
(10) Pub. No.: US 2026/0137454 A1
(43) Pub. Date: May 21, 2026

(54) SYSTEMS AND METHODS FOR PERSISTENT MARKERS

(71) Applicant: INTUITIVE SURGICAL OPERATIONS, INC., Sunnyvale, CA (US)

(72) Inventors: Gaurav Lamba, Fremont, CA (US); Sanjeev Dutta, Los Altos, CA (US); Kenneth K. Lee, Oakton, VA (US); Pourya Shirazian, Menlo Park, CA (US); Angus J. McLeod, Sunnyvale, CA (US)

(21) Appl. No.: 19/120,684

(22) PCT Filed: Oct. 11, 2023
(86) PCT No.: PCT/US2023/076522

§ 371 Date: Apr. 11, 2025

Related U.S. Application Data
(60) Provisional application No. 63/415,538, filed on Oct. 12, 2022.

(51) Int. Cl. A61B 34/10; A61B 34/20; A61B 90/00; G06T 7/579

(52) U.S. Cl. CPC A61B 34/10; A61B 34/20; A61B 90/361; A61B 90/39; G06T 7/579; A61B 2034/105; A61B 2034/2065; A61B 2090/363; A61B 2090/365; G06T 2207/10064; G06T 2207/20081; G06T 2207/30204

(57) ABSTRACT

A system may comprise a processor and a memory having computer readable instructions stored thereon. The computer readable instructions, when executed by the processor, may cause the system to receive three-dimensional primary image data from an imaging system with a field of view and generate a surface representation for a tissue surface in the field of view. The instructions may also cause the system to identify an area in the field of view with a fiducial marker, associate the fiducial marker with the surface representation, and move the surface representation and the fiducial marker in response to motion of the tissue surface in the field of view.

### CROSS-REFERENCED APPLICATIONS

This application claims priority to and benefit of U.S. Provisional Application No. 63/415,538 filed Oct. 12, 2022 and entitled “Systems and Methods for Persistent Markers,” which is incorporated by reference herein in its entirety.

### FIELD

The present disclosure is directed to systems and methods for use in robot-assisted medical procedures, and more particularly to systems and methods for generating a fiducial marker and causing the fiducial marker to persist and/or move with an anatomic image.

### BACKGROUND

Minimally invasive medical techniques are intended to reduce the amount of extraneous tissue that is damaged during diagnostic or surgical procedures, thereby reducing patient recovery time, discomfort, and harmful side effects. Such minimally invasive techniques may be performed through natural orifices in a patient anatomy or through one or more surgical incisions. Through these natural orifices or incisions, clinicians may insert medical tools to reach a target tissue location. Minimally invasive medical tools include instruments such as therapeutic instruments, diagnostic instruments, and surgical instruments. Minimally invasive medical tools may also include imaging instruments such as endoscopic instruments that provide a user with an image of a field of view within the patient anatomy.

Some minimally invasive medical tools may be robot-assisted including teleoperated, remotely operated, or otherwise computer-assisted. During a medical procedure, the clinician may view on a display an image of a field of view of the patient anatomy that may include one or more of the minimally invasive medical tools. Telestrations, visible on the display, may be generated to mark, annotate, identify, or otherwise provide graphical or alphanumeric information related to items visible in the field of view. Improved systems and methods are needed to present telestrations based on an awareness of the field of view.

### SUMMARY

The embodiments of the invention are best summarized by the claims that follow the description.

In one example, a system may comprise a processor and a memory having computer readable instructions stored thereon. The computer readable instructions, when executed by the processor, may cause the system to receive three-dimensional primary image data from an imaging system with a field of view and generate a surface representation for a tissue surface in the field of view. The instructions may also cause the system to identify an area in the field of view with a fiducial marker, associate the fiducial marker with the surface representation, and move the surface representation and the fiducial marker in response to motion of the tissue surface in the field of view.

In another example, a system may comprise a processor and a memory having computer readable instructions stored thereon. The computer readable instructions, when executed by the processor, may cause the system to receive three-dimensional primary image data from an imaging system with a field of view, receive augmented image data of the field of view from a secondary imaging modality, identify an area on a tissue surface in the augmented image data of the field of view, and generate a fiducial marker associated with the identified area.

It is to be understood that both the foregoing general description and the following detailed description are exemplary and explanatory in nature and are intended to provide an understanding of the present disclosure without limiting the scope of the present disclosure. In that regard, additional aspects, features, and advantages of the present disclosure will be apparent to one skilled in the art from the following detailed description.

### BRIEF DESCRIPTIONS OF THE DRAWINGS

FIG. 1 is a flowchart illustrating a method for associating a fiducial marker with a surface representation of a structure in a field of view, according to some examples.

FIG. 2 illustrates a display system displaying an image of a field of view according to some examples.

FIG. 3A illustrates a mesh model of a structure in a field of view, according to some examples.

FIG. 3B illustrates a continuous surface model of the structure from FIG. 3A, according to some examples.

FIG. 4A is a flowchart illustrating a method for identifying an area in a field of view with a fiducial marker, according to some examples.

FIG. 4B is a flowchart illustrating a method for identifying an area in a field of view with a fiducial marker, according to some other examples.

FIG. 5A illustrates an anatomy in a field of view of a fluorescence imaging system, according to some examples.

FIG. 5B illustrates a surface representation of the anatomy in the field of view of FIG. 5A, according to some examples.

FIG. 6A illustrates a deformation of the mesh model of FIG. 3A, according to some examples.

FIG. 6B illustrates a deformation of the surface representation of FIG. 3B, according to some examples.

FIG. 7A illustrates an anatomy in a field of view of a fluorescence imaging system, according to some examples.

FIG. 7B illustrates a surface representation of the anatomy in the field of view of FIG. 7A, according to some examples.

FIG. 7C illustrates the anatomy of the field of view of FIG. 7A with a fiducial marker, according to some examples.

FIG. 8A illustrates a surface representation of an anatomy, according to some examples.

FIG. 8B illustrates the anatomy represented by the surface representation of FIG. 8A in a field of view of a visible light imaging system, according to some examples.

FIG. 9A is a flowchart illustrating a method of associating a subsurface fiducial marker to an image or representation of a field of view, according to some examples.

FIG. 9B illustrates the anatomy of a field of view with a subsurface fiducial marker, according to some examples.

FIG. 10 illustrates a schematic view of a medical system, according to some examples.

FIG. 11 is a perspective view of a manipulator assembly of the medical system of FIG. 9, according to some examples.

FIG. 12 is a front elevation view of an operator's console in a robot-assisted medical system, according to some examples.

Embodiments of the present disclosure and their advantages are best understood by referring to the detailed description that follows. It should be appreciated that like reference numerals are used to identify like elements illustrated in one or more of the figures, wherein showings therein are for purposes of illustrating embodiments of the present disclosure and not for purposes of limiting the same.

### DETAILED DESCRIPTION

In robot-assisted medical procedures, endoscopic images of a surgical environment may provide a clinician with an image of a field of view of an anatomic region of a patient anatomy and any medical tools located in the anatomic region. During a procedure, various factors, such as the clinician's skill level, environmental distractions, shifting tissue, and aberrant anatomy, fast camera movements to a different area of the scene, and dissipation of the certain injection signal over time may detract from a clinician's ability to maintain spatial localization of structures in the anatomic region. Identifying and tracking critical structures such as vasculature, organ surfaces, ductal structures, or abnormal tissue may help the clinician maintain spatial localization of the structures during the procedure. In some situations, a clinician may physically mark the tissue (e.g., by burn marks or deposition of a marking material on the anatomy). In some situations, virtual markers that do not physically contact or change the tissue may be used to mark and track tissue. The virtual markers may include, for example, freehand telestrations drawn by a clinician, graphical symbols, alphanumeric characters, geometric shapes, or other indicators generated by a clinician, an imaging system, or a control system for a robot-assisted medical system. These virtual markers may be displayed as an overlay or integrated with the display of the field of view and may persist with various illumination modes of the field of view and may move along with the anatomic structures to which the markers are associated.

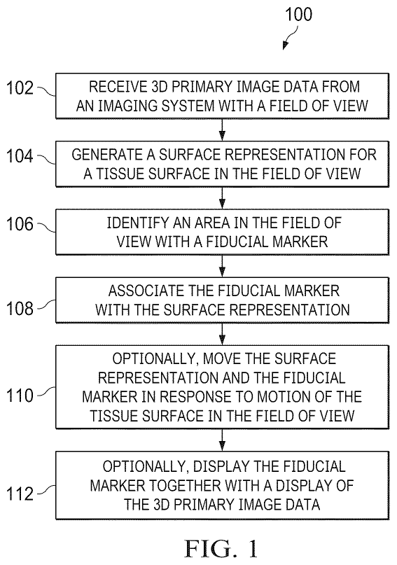

FIG. 1 is a flowchart illustrating a method 100 for associating a fiducial marker with a surface representation of a structure in a field of view. The methods described herein are illustrated as a set of operations or processes and are described with continuing reference to additional figures. Not all of the illustrated processes may be performed in all embodiments of the methods. Additionally, one or more processes that are not expressly illustrated in may be included before, after, in between, or as part of the illustrated processes. In some embodiments, one or more of the illustrated processes may be omitted. In some embodiments, one or more of the processes may be implemented, at least in part, in the form of executable code stored on non-transitory, tangible, machine-readable media that when run by one or more processors (e.g., the processing units of a control system such as control system 720) may cause the one or more processors to perform one or more of the processes. In one or more embodiments, the processes may be performed by a control system.

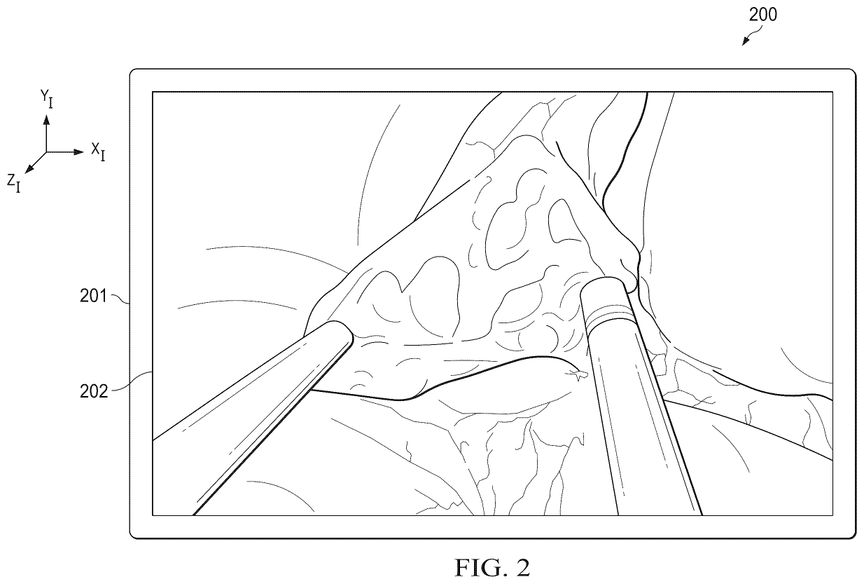

At a process 102, three-dimensional primary image data of a field of view may be received, for example, by a control system. FIG. 2, for example, provides a display system 200 including a display area 201 for displaying an image 202 of a field of view. The field of view image 202 may be generated by image data from an imaging instrument (e.g., the endoscopic imaging system 715 that may produce a stereo endoscopic imaging stream) within an anatomic environment in a patient anatomy which may include tissue surfaces, tools, suture material, and/or other naturally occurring or clinically introduced items. In this example, the image 202 may be a three-dimensional, stereoscopic image, but in other examples, the image may be a two-dimensional image. The image 202 may be, for example, an intra-operative, real-time video endoscopic image. The primary image data used to generate the image 202 may be obtained while the field of view is illuminated with visible spectrum (e.g., white) light in a standard imaging mode. The image 202 of the field of view may have an image frame of reference X_{I}, Y_{I}, Z_{I} based, for example, on a distal end of the endoscopic imaging system.

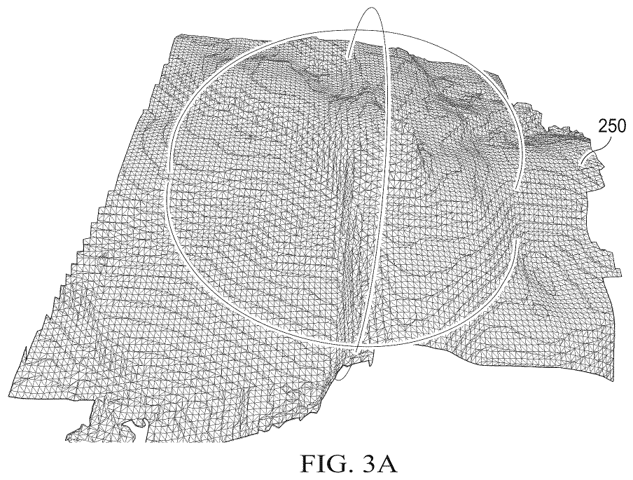

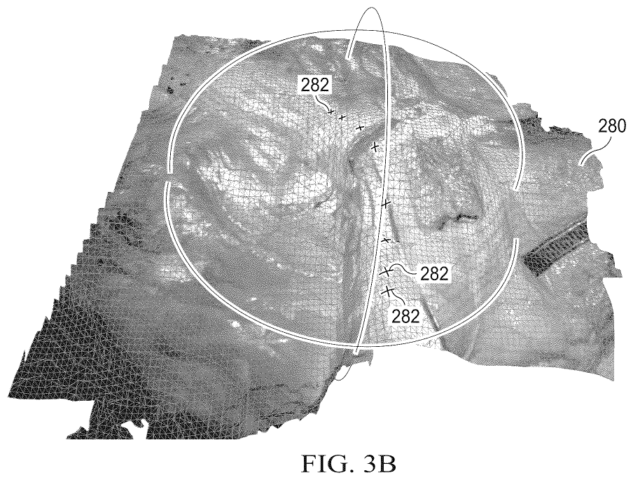

At a process 104, a surface representation may be generated for a tissue surface in the field of view. The surface representation may be, for example, a mesh model, a dense point cloud model, or a continuous surface model. A surface representation may be generated from the 3D primary image data by any of one or more surface or volumetric rendering techniques including, for example, a Simultaneous Localization and Mapping (“SLAM”) method using a mesh representation or a neural radiance field (“NeRF”) to capture the volumetric scene. FIG. 3A provides an example of a surface representation 250 that may be a mesh model generated from 3D imaging data of a field of view, and FIG. 3B provides an example of a surface representation 280 that may be a continuous surface model generated from 3D imaging data of the same field of view used to generate surface representation 250. A plurality of points 282 may be associated with locations (e.g., pixels or voxels) on the surface representation 280. The points 282 may correspond, for example, to a line or other marker created with reference to the corresponding image data, as described in greater detail below. In some examples, a plurality of surface representations 250, 280 may be generated from the same imaging data of the field of view. In some examples, the generated surface representation may be displayed, for example on the display system 200.

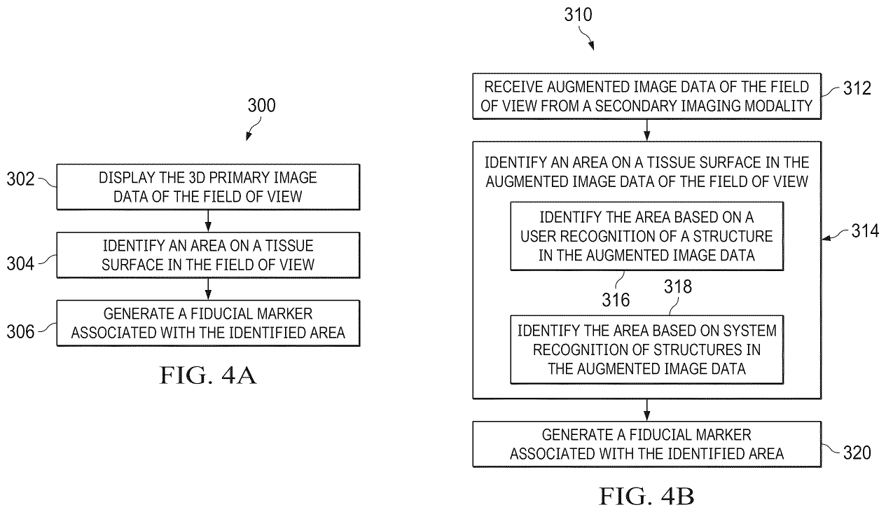

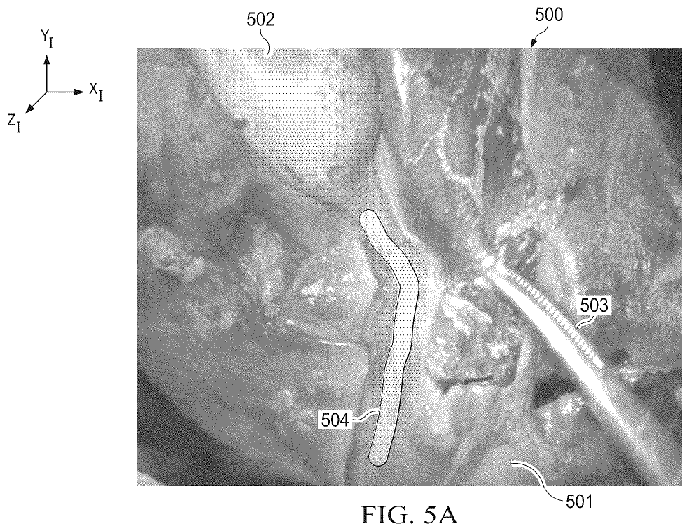

At a process 106, an area in the field of view may be identified with a fiducial marker. As an example, FIG. 4A illustrates a method 300 for identifying an area with a fiducial marker. At a process 302, the 3D primary image data of the field of view may be generated from may be displayed on a display system. At a process 304, an area on the surface representation of the field of view may be identified. For example, a user may interact with the displayed image via a user interface (e.g., a touch screen, a mouse, a keyboard, a manipulator of a robot-assisted medical system) to indicate an area in the field of view. The area may be, for example, an area including structures of interest to the procedure such as vasculature, organ surfaces, ductal structures, and/or tumors or other abnormal tissue. The area may signify procedurally relevant tissue including boundaries, proposed treatment locations, proposed lines of resection, or other tissue areas that may be involved in or avoided during the procedure. In some examples, the area may be identified based on a computational assessment by, for example, a recognition system or control system based on image processing, reference to models of similar anatomic areas, or other analysis of the 3D primary image data. At a process 306, a fiducial marker may be generated. The fiducial marker may be a freehand telestration (e.g., drawn by a user), graphical symbols, alphanumeric characters, geometric shapes, or other indicators. Fiducial marker may be marked on the surface of the mesh (i.e. tissue/organ surface) but may also be located above or underneath the surface. The markers may be registered to the nearby area of the surface via a vector field that represents the spatial relationship between the surface of the markers.

As another example of a method for identifying an area with a fiducial marker, FIG. 4B illustrates a method 310. At a process 312, augmented image data of the field of view may be received from a secondary imaging modality. For example, augmented image data may be generated from a secondary, co-registered imaging modality such as an imaging modality using additional or alternative light wavelengths or such as an image processing mode that is different from a primary image processing mode. Co-registered imaging modalities may include, for example fluorescence imaging (e.g., “firefly” fluorescence imaging available on some systems provided by Intuitive Surgical, Inc.), hyperspectral imaging, laser speckle contrast imaging, oxygenated/deoxygenated hemoglobin concentration imaging, Ramen spectroscopy, and/or other analytic light imaging modalities.

Images from a co-registered imaging system may appear quite different from the color images from the visible light spectrum imaging system. The tracking/mapping system (e.g. SLAM) may handle the two different types of images by using color-invariant features. For example, all the images may be changed to grey-scale to allow focus on the features/shapes and not the color of the image. This may allow the user to create markers in the fluorescence imaging and then track the created markers in the visible light imaging.

In some examples, the augmented image data of the field of view may be image data from a near-infrared fluorescence imaging system. The fluorescence imaging system may be integrated with a visible light endoscopic imaging system (e.g. integrated into the imaging system 715) and may provide fluorescent imaging to visualize, for example, vessels, blood flow, bile ducts, and related tissue perfusion. To generate augmented image data and assess tissue with a fluorescence imaging system, a fluorescence dye (e.g., indocyanine green “ICG”) may be injected intravenously into a patient. The ICG may bind to plasma proteins, such as albumin, in the blood and emit an infrared signal when excited by laser light (e.g. at 803 nm wavelength) in situ. The laser light may be emitted by an infrared excitation laser separate from a visible light endoscope or may be emitted by an illuminator light emitting diode with an infrared excitation laser that is integrated into an endoscope that also images with visible light. The glowing fluorescing dye may be detected by a control system and software algorithms may be used to colorize the fluorescence signal for display on the display system. The control system may allow a user to switch between visible light and fluorescence imaging modes from an operator console. As a more specific example, during a cholecystectomy, intravenously provided ICG may concentrate in the patient's bile and may become fluorescent under near infrared spectrum light, allowing a clinician to identify bile duct structures critical in the removal of the gall bladder. Over time, however, the ICG may dissipate, making continued tracking of the bile ducts difficult. Marking the anatomic structures with persistent markers at peak or near peak fluorescence may allow the identification to continue even after the dye has dissipated or if high levels of fluorescence in background structures obscure the identified areas.

At a process 314, an area on a tissue surface may be identified in the augmented image data of the field of view. The identification can be performed by a clinician or other user viewing the augmented image data or by a control system using image analysis techniques to recognize structures in the augmented data. For example, at a process 316, a user viewing the augmented image data on a display may recognize, and thus identify, areas of the field of view that correspond to areas of peak fluorescence. Alternatively or additionally, at a process 318, a recognition system (e.g. an image processing system of the control system 720 or in communication with the control system 720) may identify areas of peak fluorescence. In some examples, the recognition system may use an artificial intelligence system and machine learning to recognize structures based on characteristics of the augmented image data. In some examples, the recognition system may provide a recommended identification that may be confirmed, rejected, or modified by a user. The structures of interest identified at process 314 may be dependent on the type of procedure. For example, for a cholecystectomy procedure, structures such as the bile duct, the cystic artery, and the cystic duct may be identified. For a prostatectomy, a prostate tumor may be identified. For a urological or gynecological procedure, the ureter may be identified. For a colorectal surgery, areas of high perfusion may be identified.

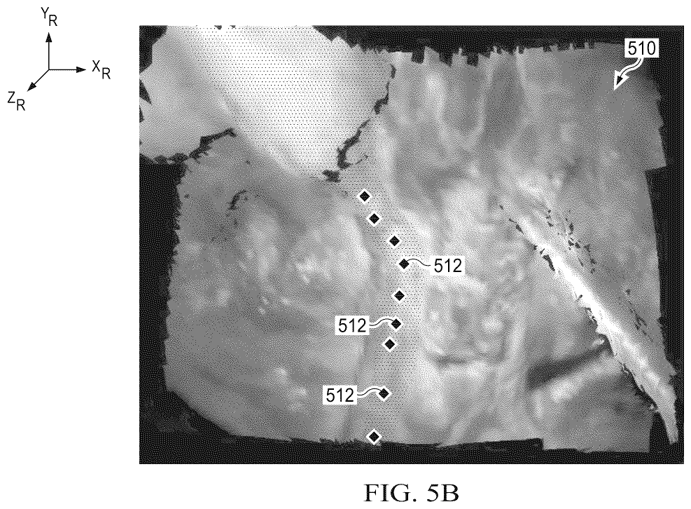

At a process 320, a fiducial marker may be generated based on the identified area in the augmented image data. FIG. 5A illustrates a field of view 500 of a patient anatomy captured by a fluorescence imaging system with an image frame of reference X_{I}, Y_{I}, Z_{I}. Structures in the field of view 500 may include tissue 501 and a tool 503. Tissue structures, such as vessels and perfused tissue, in an area 502 may glow under near infrared light provided by the imaging system. To track the fluorescing structures, even after the fluorescent dye begins to fade, the user may create a fiducial marker 504, such as a curved line, that tracks the vessel structure. The line may be generated manually with a user interface device such as a touchscreen, a stylus, a mouse, or a manipulator of the robot-assisted medical system. In some examples, the user interface device may constrain input to two-dimensions (e.g., X and Y directions). In some examples, if the area is identified based on a recognition system, the control system may generate the line 504 to correspond to the recognized pattern of the peak fluorescence.

With reference again to FIG. 1, the method 100 may continue with a process 108 wherein the fiducial marker is associated with the surface representation. For example the fiducial marker may be placed on a three-dimensional surface representation (e.g., a mesh model or a continuous surface model) of the area of the anatomy in the field of view. Portions of the fiducial marker may be placed at three-dimensional locations that correspond to the identified area. FIG. 5B, for example, illustrates a surface representation 510 with a frame of reference X_{R}, Y_{R}, Z_{R} that corresponds to (e.g., registered to) and represents the tissue in the field of view 500. The surface representation 510 may be generated based on a depth map of the structures in the field of view 500. A sequence or cloud of points corresponding to the graphic marker 504 may be projected as a sequence or cloud of point 512 on the surface representation 510 to mark the area 502. Each of the points 512 may correspond to an X, Y, Z coordinate on the surface representation 510. Thus, even though the marker may be generated with a two-dimensional constrained user interface, each point 512 may have a Z depth value along the surface representation 510. In some examples, the marker 504 may be sampled at a fixed pixel spacing to generate the points 512 that are projected to the surface representation 510. For example, using a SLAM process, a depth map and stereo image calibration may be used to place the points 512 on a 3D vector map.

In some examples, the marker 504 may be rendered in the image frame of reference as a two-dimensional marker that extends over the identified area. In other examples, the marker 504 may be rendered in the image frame of reference as a three-dimensional marker that is contoured to the surface of the tissue in the field of view, based on the registered surface representation 510 and the projected points 512. The marker 504 may be displayed as a three-dimensional overlay on the surface field of view 500 by projecting the marker in stereoscopic images. In various examples, the three-dimensional marker may be displayed as an undulating graphical line (e.g. continuous or dotted), a series of discrete symbols, or as alphanumeric/character text. The marker may be drawn freehand or may be selected from pre-established graphic options. Properties of the marker 504 may be altered based on user selection, proximity of tools in the field of view, or other user or system selection criteria. For example the display of the marker may be turned on or off, the color of the marker may be changed, and/or the style of the marker (e.g., continuous or dotted line) may be altered.

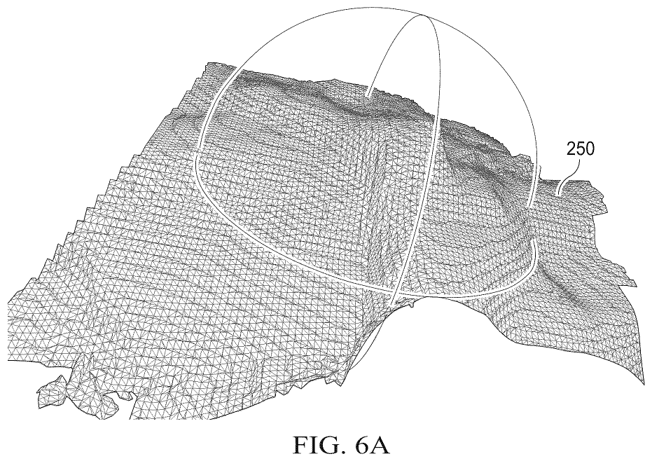

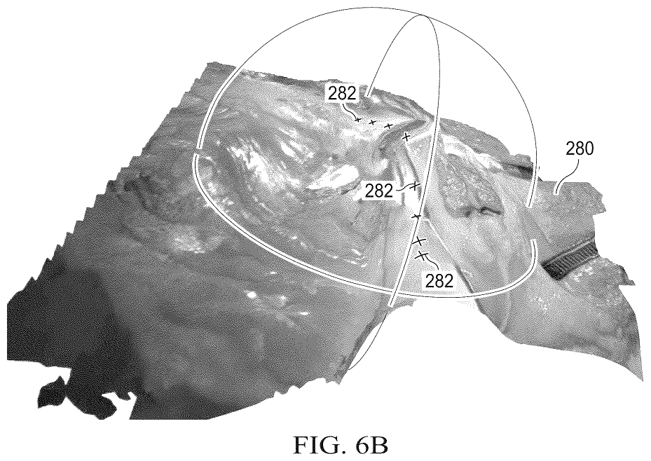

At an optional process 110, the surface representation and the fiducial marker may move in response to a change of the tissue surface in the field of view. Tissue change may be due to motion, for example, to breathing, cardiac activity, surgical tool intervention, or other forces during a medical procedure. The changed appearance of the tissue in the field of view may also or alternatively result from a change in the position and/or orientation of the imaging system (e.g. the endoscope). As shown in FIGS. 6A and 6B, the surface representations 250, 280 may change (relative to their configuration in FIGS. 3A and 3B) based on the motion of the tissue they represent as captured in the field of view. For example, surface representation may be updated at interactive frame rates as part of a SLAM process that uses a real-time 3D vector map to track tissue deformation and endoscope motion. The change of the tissue in the field of view may result from tissue motion or from a change is position and/or orientation of the imaging system. As the surface representation 280 moves, the markers or points 282 associated with locations, pixels, or voxels on the surface representation move with the representation. Thus, as the tissue associated with the points 282 is displaced, stretched, occluded, dissected, or otherwise changed, the points 282 and the marker in the image frame of reference that is associated with the points is likewise changed. By using the information from SLAM, markers (or any type of drawings or annotations) may be placed during the deformation of the surface and/or during camera motion. This may allow for real-time placement and visual feedback during the initial digital fiducial marking. In some examples, the SLAM process may be a deformable SLAM process that performs simultaneous localization and mapping for deformable or non-rigid surfaces, volumes, structures, and environments.

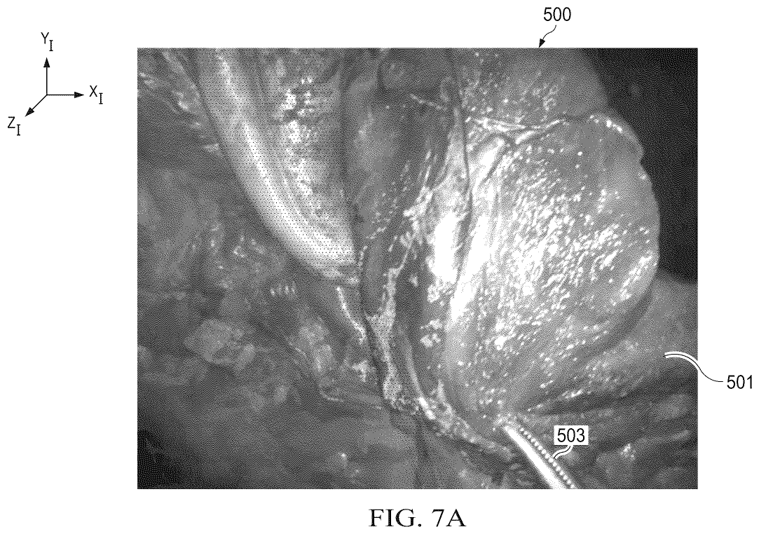

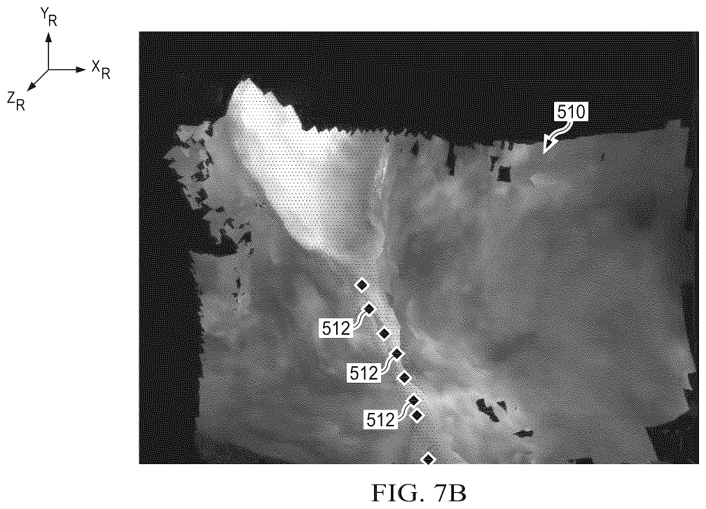

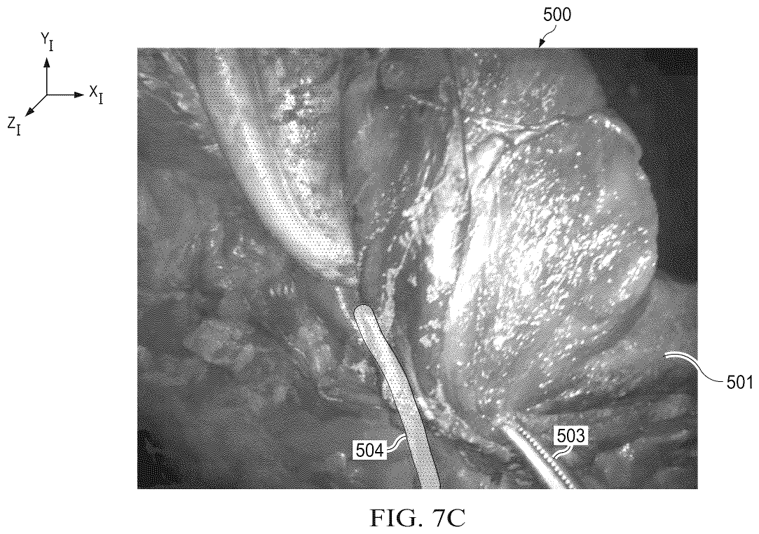

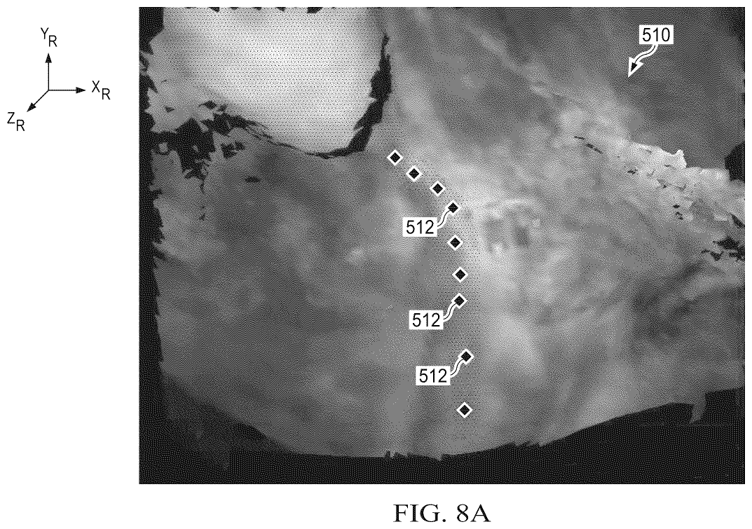

As the surface representation and the points associated with the fiducial marker change, the display of the fiducial marker may also be changed. For example, FIG. 7A illustrates the field of view 500 with the tissue 501 and the tool 503 in different positions and orientations, as compared to FIG. 5A. FIG. 7B illustrates the surface representation 510 changed in accordance with the change in field of view 500 in FIG. 7A. The location of the points 512 in the surface representation frame of reference, which are associated with locations on the surface representation 510, are likewise changed. FIG. 7C illustrates the field of view 500 with the fiducial marker 504 displayed in an updated configuration that corresponds to the change in the points 512. Thus, as the structures in the field of view 500 move, the fiducial marker 504 persists and moves with the identified anatomic structure. Accordingly, the associated critical anatomic structure may be tracked, even as the fluorescing dye dissipates. Tracking the fiducial marker 504 may include updating the displacement, orientation, configuration, and/or deformation of the marker as the associated pixels, voxels, or other associated graphic elements move. The marker 504 may stretch or bend such that a distal portion of the marker moves relative to a proximal portion of the marker as the associated tissue moves. The surface representation 510, and consequently the location of the points 512 and the marker 504, may be updated using surface and/or volumetric rendering techniques such as SLAM applied to the stereo endoscopic video stream. The SLAM process may include a deformable SLAM process that process may be used to produce updated surface and volumetric renderings in response to image system motion or tissue deformation while tracking subsurface fiducial markers relative to the updated renderings.

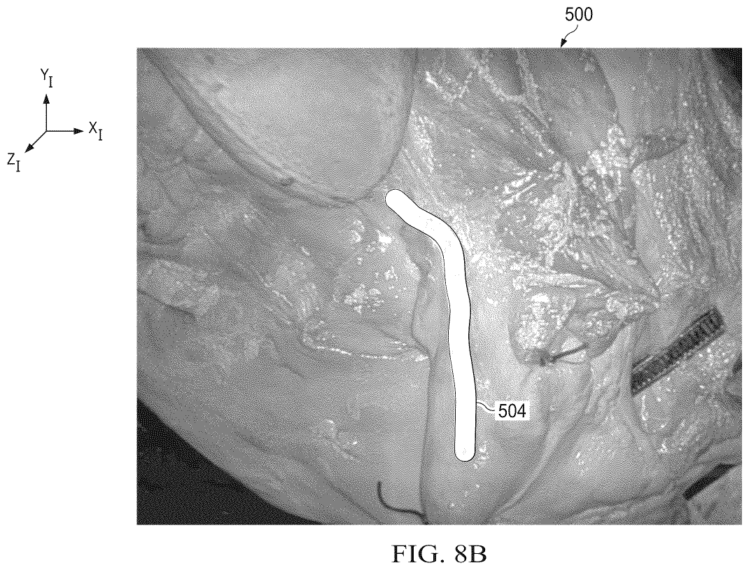

At an optional process 112, the fiducial marker may be displayed with other co-registered image data. For example, a fiducial marker may be displayed with the 3D primary image data. FIG. 8A illustrates the surface representation 510 and the points 512 in a revised configuration. FIG. 8B illustrates the field of view 500 as viewed with only visible light and no near infrared light to highlight critical structures. The line 504 corresponding to the points 512 may be displayed as a three-dimensional overlay or otherwise integrated with the visible light illuminated field of view 500 based upon the registration of the field of view 500 in the image reference frame with the surface representation 510 in the representation reference frame. The three-dimensional nature of the marker 504 may be achieved by presenting stereoscopic images of the marker and the visible light illuminated field of view to the user. Thus, the fiducial marker 504 may persist to allow a clinician to track the critical structure even without the fluorescence image.

In some examples, the endoscopic camera may move such that the new camera field of view does not include the structure marked with the fiducial marker. In this example, the rendering technique (e.g., SLAM) may create a map of the entire surgical area or just the critical areas. Thus, when the camera moves back to visualize the field of view where the digital fiducial was initially drawn, the system will automatically recognize and re-localize to that area and display the digital fiducial at the correct location.

In some examples, a secondary imaging modality provides subsurface image data and selected structure images, telestrations, labels, markers, or other information associated with subsurface structures may be displayed with primary image data or surface representations of the primary image data. In some examples, the information associated with the subsurface structures may persist with the displayed primary image data as the primary imaging system moves.

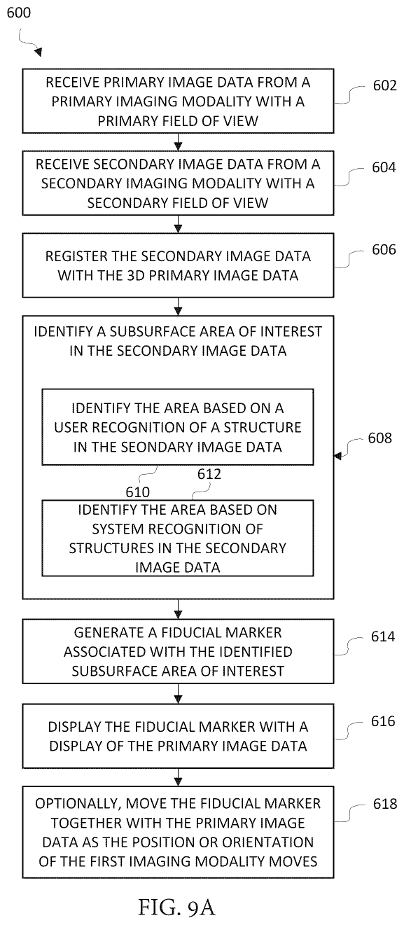

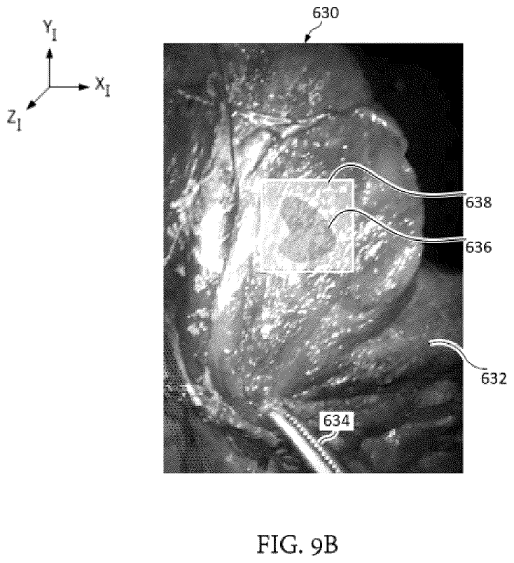

FIG. 9A is a flowchart illustrating a method 600 of associating a fiducial marker corresponding to a subsurface structure to an image or representation of a field of view. At a process 602, primary image data of a field of view may be received, for example, by a control system. The primary image data may be received from a primary imaging modality and may be three-dimensional image data. This process may be substantially similar to process 102 described above. At a process 604, secondary image data may be received from a secondary imaging modality having a secondary field of view. For example, image data may be generated from a secondary imaging modality such as an imaging modality using high frequency acoustic waves (e.g., ultrasound imaging) to image subsurface tissue structures. The secondary image data may, for example, provide subsurface images of tissues within the field of view of the primary image data. In some examples, the secondary image data may be generated prior to receiving the primary image data. For example, a prior to an endoscopic imaging procedure, an ultrasound scan may be performed to generate the secondary image data. In other examples, the secondary image data may be ultrasound imaging data generated by an ultrasound probe extended within or along an endoscope for generating the primary image data.

At a process 606, the secondary image data may be registered to the primary image data. Registering the primary and secondary image data sets may include spatially aligning the two image datasets or transforming one of the datasets to the coordinate system of the other using any of a variety of registration techniques including feature matching and/or image system tracking. In some examples, the registration process 606 may be performed at a different stage of the method, for example after process 608 or after process 614.

At a process 608, a subsurface area may be identified in the secondary imaging data. For example, the area identified may be a structure such as a tumor, a boundary of a tumor, an anatomic duct (e.g., a ureter), vasculature or another anatomic structure of interest or relevance to a medical procedure that is located below the surface of the tissue visible in the field of view of the primary image data. The secondary imaging data, such as ultrasound imaging data, may visualize the subsurface structures. The identification can be performed by a clinician or another user viewing the secondary image data or by a control system using image analysis techniques to recognize structures in the secondary image data. For example, at a process 610, a user viewing the secondary image data on a display may recognize, and thus identify, areas that correspond to a structure of interest. Alternatively or additionally, at a process 612, a recognition system (e.g. an image processing system of the control system 720 or in communication with the control system 720) may identify areas of interest. In some examples, the recognition system may use an artificial intelligence system and machine learning to recognize structures based on characteristics of the secondary image data. In some examples, the recognition system may provide a recommended identification that may be confirmed, rejected, or modified by a user. The structures of interest identified at process 608 may be dependent on the type of procedure. For a prostatectomy, a prostate tumor boundary may be identified. For a urological or gynecological procedure, the ureter may be identified.

At a process 614, a fiducial marker may be generated based on the identified area in the secondary image data. The fiducial marker may be, for example, a telestration marking a tissue boundary, outlining a structure, indicating a fluid flow, or otherwise marking an area of interest. In some examples, the fiducial marker may be a portion of the secondary image data, such as a portion depicting a tumor. In some examples, the fiducial marker may be a flag or a label indicating an area of clinical intervention or interest. In some examples, the fiducial marker may include numerical or textual characters, symbols, lines, shapes, or other graphical representations. In some examples, the fiducial marker may be generated manually with a user interface device such as a touchscreen, a stylus, a mouse, or a manipulator of the robot-assisted medical system.

At a process 616, the fiducial marker may be displayed with the primary image data. For example, the subsurface fiducial marker may be displayed as an underlayer below a see-through window, a semi-transparent window, or other type of opaque or visually distinct rendering of the tissue surface in the field of view of the primary image data. In other examples, the fiducial marker may be integrated with the primary image day, overlayed on the primary image data or placed on a three-dimensional surface representation (e.g., a mesh model or a continuous surface model) of the area of the anatomy in the field of view generated from the primary image data. FIG. 9B, for example, illustrates a field of view 630 captured by primary image data. The field of view 630 includes tissue 632 and a tool 634. A fiducial marker 636 associated with an identified subsurface area of interest from secondary ultrasound imaging data is shown with the field of view 630 of the primary image data. In this example, the fiducial marker 636 may mark the border or margin of a tumor and may be visible as an underlayer below a semi-transparent surface 638 of the tissue 632 in the field of view 630.

At the process 618, the fiducial marker and may persist as the primary imaging system changes positions or orientations and may move with the tissue in the field of view or may with the field of view as the anatomic structures, with which the markers are associated, move within the field of view. Tissue motion may be due, for example, to breathing, cardiac activity, surgical tool intervention, or other forces during a medical procedure. The tissue in the field of view may also or alternatively move as a result of a change in the position and/or orientation of the imaging system (e.g. the endoscope). For example, fiducial marker may be added to and update at interactive frame rates as part of a SLAM process that uses a real-time 3D vector map to track tissue deformation and endoscope motion. The change of the tissue in the field of view may result from tissue motion or from a change is position and/or orientation of the imaging system. As the tissue or surface representation of the tissue moves, the fiducial markers associated with locations, pixels, or voxels representing the tissue may also have a corresponding motion. For example, a deformable SLAM process may be used to produce updated surface and volumetric renderings in response to image system motion or tissue deformation while tracking subsurface fiducial markers. Thus, as the tissue associated with the fiducial marker is displaced, stretched, occluded, dissected, or otherwise changed, the fiducial marker in the field of view is likewise changed. As the surface representation and the points associated with the fiducial marker change, the display of the fiducial marker may also be changed. Thus, as the structures in the field of view 630 move, the fiducial marker 636 persists and moves with the overlayed anatomic structure. Accordingly, the associated critical anatomic structure may be tracked, even in the absence of current secondary image data. Tracking the fiducial marker 636 may include updating the displacement, orientation, configuration, and/or deformation of the marker as the associated pixels, voxels, or other associated graphic elements move. The marker 636 may stretch or bend. The fiducial marker 636 may be updated using surface and/or volumetric rendering techniques such as the SLAM applied to the stereo endoscopic video stream.

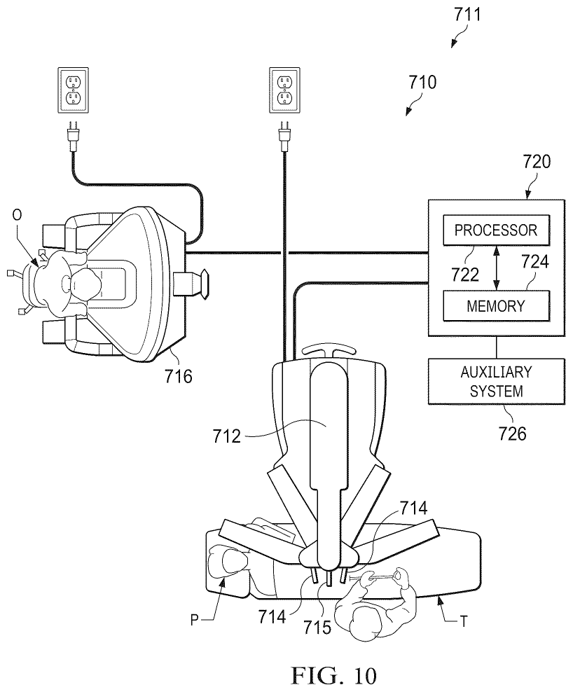

FIGS. 10-12 together provide an overview of a medical system 710 that may be used in, for example, medical procedures including diagnostic, therapeutic, or surgical procedures. The fiducial marker generation and tracking examples provided above may be used in the context of the medical system 710. The medical system 710 is located in a medical environment 711. The medical environment 711 is depicted as an operating room in FIG. 10. In other embodiments, the medical environment 711 may be an emergency room, a medical training environment, a medical laboratory, or some other type of environment in which any number of medical procedures or medical training procedures may take place. In still other embodiments, the medical environment 711 may include an operating room and a control area located outside of the operating room.

In one or more embodiments, the medical system 710 may be a robot-assisted medical system that is under the teleoperational control of an operator (e.g., a surgeon, a clinician, a physician, etc.). In alternative embodiments, the medical system 710 may be under the partial control of a computer programmed to perform the medical procedure or sub-procedure. In still other alternative embodiments, the medical system 710 may be a fully automated medical system that is under the full control of a computer programmed to perform the medical procedure or sub-procedure with the medical system 710. One example of the medical system 710 that may be used to implement the systems and techniques described in this disclosure is the da Vinci® Surgical System manufactured by Intuitive Surgical, Inc. of Sunnyvale, California.

As shown in FIG. 10, the medical system 710 generally includes an assembly 712, which may be mounted to or positioned near an operating table T on which a patient P is positioned. The assembly 712 may be referred to as a patient side cart, a surgical cart, or a surgical robot. In one or more embodiments, the assembly 712 may be a teleoperational assembly. The teleoperational assembly may be referred to as, for example, a teleoperational arm cart. A medical instrument system 714 and an endoscopic imaging system 715 are operably coupled to the assembly 712. An operator input system 716 allows an operator O or other type of clinician to view images of or representing the surgical site and to control the operation of the medical instrument system 714 and/or the endoscopic imaging system 715.

The medical instrument system 714 may comprise one or more medical instruments. In embodiments in which the medical instrument system 714 comprises a plurality of medical instruments, the plurality of medical instruments may include multiple of the same medical instrument and/or multiple different medical instruments. Similarly, the endoscopic imaging system 715 may comprise one or more endoscopes. In the case of a plurality of endoscopes, the plurality of endoscopes may include multiple of the same endoscope and/or multiple different endoscopes.

The operator input system 716 may be located at an operator's control console, which may be located in the same room as operating table T. In some embodiments, the operator O and the operator input system 716 may be located in a different room or a completely different building from the patient P. The operator input system 716 generally includes one or more control device(s) for controlling the medical instrument system 714. The control device(s) may include one or more of any number of a variety of input devices, such as hand grips, joysticks, trackballs, data gloves, trigger-guns, foot pedals, hand-operated controllers, voice recognition devices, touch screens, body motion or presence sensors, and other types of input devices.

In some embodiments, the control device(s) will be provided with the same degrees of freedom as the medical instrument(s) of the medical instrument system 714 to provide the operator with telepresence, which is the perception that the control device(s) are integral with the instruments so that the operator has a strong sense of directly controlling instruments as if present at the surgical site. In other embodiments, the control device(s) may have more or fewer degrees of freedom than the associated medical instruments and still provide the operator with telepresence. In some embodiments, the control device(s) are manual input devices that are movable with six degrees of freedom, and which may also include an actuatable handle for actuating instruments (for example, for closing grasping jaw end effectors, applying an electrical potential to an electrode, delivering a medicinal treatment, and actuating other types of instruments).

The assembly 712 may support and manipulate the medical instrument system 714 while the operator O views the surgical site through the operator input system 716. An image of the surgical site may be obtained by the endoscopic imaging system 715, which may be manipulated by the assembly 712. The assembly 712 may comprise endoscopic imaging systems 715 and may similarly comprise multiple medical instrument systems 714 as well. The number of medical instrument systems 714 used at one time will generally depend on the diagnostic or surgical procedure to be performed and on space constraints within the operating room, among other factors. The assembly 712 may include a kinematic structure of one or more non-servo controlled links (e.g., one or more links that may be manually positioned and locked in place, generally referred to as a set-up structure) and a manipulator. When the manipulator takes the form of a teleoperational manipulator, the assembly 712 is a teleoperational assembly. The assembly 712 includes a plurality of motors that drive inputs on the medical instrument system 714. In an embodiment, these motors move in response to commands from a control system (e.g., control system 720). The motors include drive systems which when coupled to the medical instrument system 714 may advance a medical instrument into a naturally or surgically created anatomical orifice. Other motorized drive systems may move the distal end of said medical instrument in multiple degrees of freedom, which may include three degrees of linear motion (e.g., linear motion along the X, Y, Z Cartesian axes) and three degrees of rotational motion (e.g., rotation about the X, Y, Z Cartesian axes). Additionally, the motors may be used to actuate an articulable end effector of the medical instrument for grasping tissue in the jaws of a biopsy device or the like. Medical instruments of the medical instrument system 714 may include end effectors having a single working member such as a scalpel, a blunt blade, an optical fiber, or an electrode. Other end effectors may include, for example, forceps, graspers, scissors, or clip appliers.

The medical system 710 also includes a control system 720. The control system 720 includes at least one memory 724 and at least one processor 722 for effecting control between the medical instrument system 714, the operator input system 716, and other auxiliary systems 726 which may include, for example, imaging systems, image recognition systems, audio systems, fluid delivery systems, display systems, illumination systems, steering control systems, irrigation systems, and/or suction systems. A clinician may circulate within the medical environment 711 and may access, for example, the assembly 712 during a set up procedure or view a display (e.g., display system 200) of the auxiliary system 726 from the patient bedside.

Though depicted as being external to the assembly 712 in FIG. 9, the control system 720 may, in some embodiments, be contained wholly within the assembly 712. The control system 720 also includes programmed instructions (e.g., stored on a non-transitory, computer-readable medium) to implement some or all of the methods described in accordance with aspects disclosed herein. While the control system 720 is shown as a single block in the simplified schematic of FIG. 9, the control system 720 may include two or more data processing circuits with one portion of the processing optionally being performed on or adjacent the assembly 712, another portion of the processing being performed at the operator input system 716, and the like.

Any of a wide variety of centralized or distributed data processing architectures may be employed. Similarly, the programmed instructions may be implemented as a number of separate programs or subroutines, or they may be integrated into a number of other aspects of the systems described herein, including teleoperational systems. In one embodiment, the control system 720 supports wireless communication protocols such as Bluetooth, IrDA, HomeRF, IEEE 802.11, DECT, and Wireless Telemetry.

In some embodiments, control system 720 may include one or more servo controllers that receive force and/or torque feedback from the medical instrument system 714. Responsive to the feedback, the servo controllers transmit signals to the operator input system 716. The servo controller(s) may also transmit signals instructing assembly 712 to move the medical instrument system(s) 714 and/or endoscopic imaging system 715 which extend into an internal surgical site within the patient body via openings in the body. Any suitable conventional or specialized servo controller may be used. A servo controller may be separate from, or integrated with, assembly 712. In some embodiments, the servo controller and assembly 712 are provided as part of a teleoperational arm cart positioned adjacent to the patient's body.

The control system 720 can be coupled with the endoscopic imaging system 715 and can include a processor to process captured images for subsequent display, such as to an operator on the operator's control console, or on another suitable display located locally and/or remotely. For example, where a stereoscopic endoscope is used, the control system 720 can process the captured images to present the operator with coordinated stereo images of the surgical site. Such coordination can include alignment between the opposing images and can include adjusting the stereo working distance of the stereoscopic endoscope.

In alternative embodiments, the medical system 710 may include more than one assembly 712 and/or more than one operator input system 716. The exact number of assemblies 712 will depend on the surgical procedure and the space constraints within the operating room, among other factors. The operator input systems 716 may be collocated or they may be positioned in separate locations. Multiple operator input systems 716 allow more than one operator to control one or more assemblies 712 in various combinations. The medical system 710 may also be used to train and rehearse medical procedures.

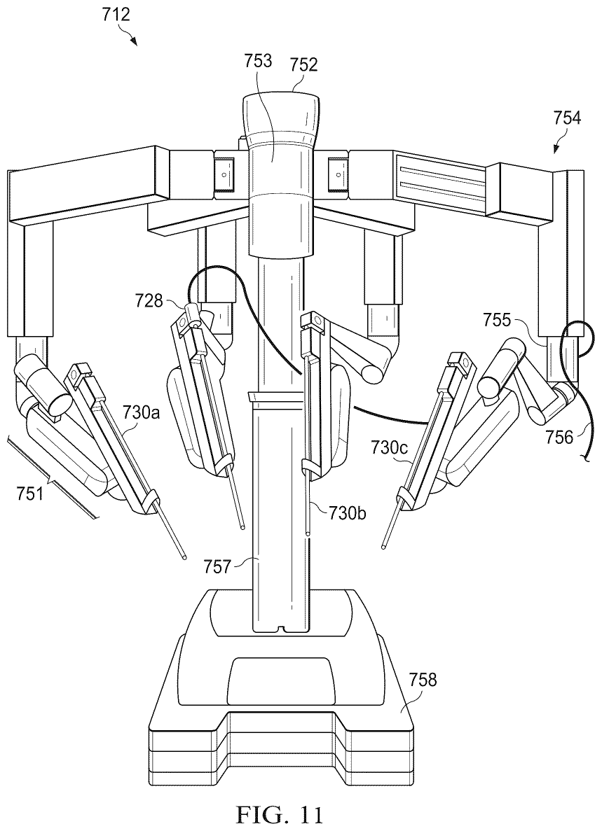

FIG. 11 is a perspective view of one embodiment of an assembly 712 which may be referred to as a patient side cart, surgical cart, teleoperational arm cart, manipulator assembly or surgical robot. The assembly 712 shown provides for the manipulation of three surgical tools 730a, 730b, and 730c (e.g., medical instrument systems 714) and an imaging device 728 (e.g., endoscopic imaging system 715), such as a stereoscopic endoscope used for the capture of images of the site of the procedure. The imaging device may transmit signals over a cable 756 to the control system 720. Manipulation is provided by teleoperative mechanisms having a number of joints. The imaging device 728 and the surgical tools 730a-c can be positioned and manipulated through incisions in the patient so that a kinematic remote center is maintained at the incision to minimize the size of the incision. Images of the surgical site can include images of the distal ends of the surgical tools 730a-c when they are positioned within the field of view of the imaging device 728.

The assembly 712 includes a drivable base 758. The drivable base 758 is connected to a telescoping column 757, which allows for adjustment of the height of arms 754. The arms 754 may include a rotating joint 755 that both rotates and moves up and down. Each of the arms 754 may be connected to an orienting platform 753. The arms 754 may be labeled to facilitate trouble shooting. For example, each of the arms 754 may be emblazoned with a different number, letter, symbol, other identifier, or combinations thereof. The orienting platform 753 may be capable of 760 degrees of rotation. The assembly 712 may also include a telescoping horizontal cantilever 752 for moving the orienting platform 753 in a horizontal direction.

In the present example, each of the arms 754 connects to a manipulator arm 751. The manipulator arms 751 may connect directly to a medical instrument, e.g., one of the surgical tools 730a-c. The manipulator arms 751 may be teleoperable. In some examples, the arms 754 connecting to the orienting platform 753 may not be teleoperable. Rather, such arms 754 may be positioned as desired before the operator O begins operation with the teleoperative components. Throughout a surgical procedure, medical instruments may be removed and replaced with other instruments such that instrument to arm associations may change during the procedure.

Endoscopic imaging systems (e.g., endoscopic imaging system 715 and imaging device 728) may be provided in a variety of configurations including rigid or flexible endoscopes. Rigid endoscopes include a rigid tube housing a relay lens system for transmitting an image from a distal end to a proximal end of the endoscope. Flexible endoscopes transmit images using one or more flexible optical fibers. Digital image-based endoscopes have a “chip on the tip” design in which a distal digital sensor such as a one or more charge-coupled device (CCD) or a complementary metal oxide semiconductor (CMOS) device store image data. Endoscopic imaging systems may provide two- or three-dimensional images to the viewer. Two-dimensional images may provide limited depth perception. Three-dimensional stereo endoscopic images may provide the viewer with more accurate depth perception. Stereo endoscopic instruments employ stereo cameras to capture stereo images of the patient anatomy. An endoscopic instrument may be a fully sterilizable assembly with the endoscope cable, handle and shaft all rigidly coupled and hermetically sealed.

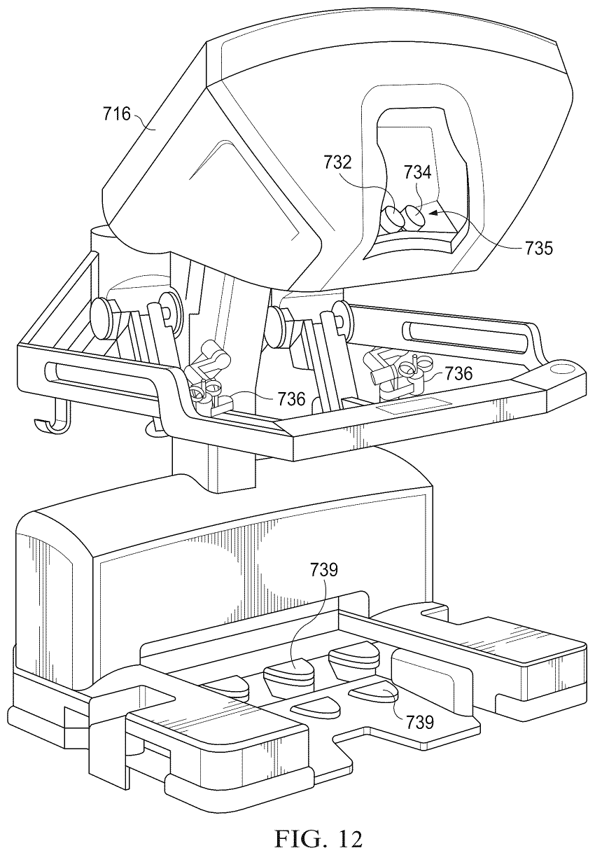

FIG. 12 is a perspective view of an embodiment of the operator input system 716 at the operator's control console. The operator input system 716 includes a display system (e.g. display system 200) with a left eye display 732 and a right eye display 734 for presenting the operator O with a coordinated stereo view of the surgical environment that enables depth perception. The left and right eye displays 732, 732 may be components of a display system 735 (e.g., the display system 200). In other embodiments, the display system 735 may include one or more other types of displays. The display system 735 may present images captured, for example, by the imaging system 715 to display the endoscopic field of view to the operator. The endoscopic field of view may be augmented by virtual or synthetic menus, indicators, and/or other graphical or textual information to provide additional information to the viewer.

The operator input system 716 further includes one or more input control devices 736, which in turn cause the assembly 712 to manipulate one or more instruments of the endoscopic imaging system 715 and/or medical instrument system 714. The input control devices 736 can provide the same degrees of freedom as their associated instruments to provide the operator O with telepresence, or the perception that the input control devices 736 are integral with said instruments so that the operator has a strong sense of directly controlling the instruments. To this end, position, force, and tactile feedback sensors (not shown) may be employed to transmit position, force, and tactile sensations from the medical instruments, e.g., surgical tools 730a-c, or imaging device 728, back to the operator's hands through the input control devices 736. Input control devices 739 are foot pedals that receive input from a user's foot. Aspects of the operator input system 716, the assembly 712, and the auxiliary systems 726 may be adjustable and customizable to meet the physical needs, skill level, or preferences of the operator O.

Elements described in detail with reference to one embodiment, implementation, or application optionally may be included, whenever practical, in other embodiments, implementations, or applications in which they are not specifically shown or described. For example, if an element is described in detail with reference to one embodiment and is not described with reference to a second embodiment, the element may nevertheless be claimed as included in the second embodiment. Thus, to avoid unnecessary repetition in the following description, one or more elements shown and described in association with one embodiment, implementation, or application may be incorporated into other embodiments, implementations, or aspects unless specifically described otherwise, unless the one or more elements would make an embodiment or implementation non-functional, or unless two or more of the elements provide conflicting functions.

Any alterations and further modifications to the described devices, systems, instruments, methods, and any further application of the principles of the present disclosure are fully contemplated as would normally occur to one skilled in the art to which the disclosure relates. In particular, it is fully contemplated that the features, components, and/or steps described with respect to one embodiment may be combined with the features, components, and/or steps described with respect to other embodiments of the present disclosure. In addition, dimensions provided herein are for specific examples and it is contemplated that different sizes, dimensions, and/or ratios may be utilized to implement the concepts of the present disclosure. To avoid needless descriptive repetition, one or more components or actions described in accordance with one illustrative embodiment can be used or omitted as applicable from other illustrative embodiments. For the sake of brevity, the numerous iterations of these combinations will not be described separately.

Various systems and portions of systems have been described in terms of their state in three-dimensional space. As used herein, the term “position” refers to the location of an object or a portion of an object in a three-dimensional space (e.g., three degrees of translational freedom along Cartesian X, Y, Z coordinates). As used herein, the term “orientation” refers to the rotational placement of an object or a portion of an object (three degrees of rotational freedom—e.g., roll, pitch, and yaw). As used herein, the term “pose” refers to the position of an object or a portion of an object in at least one degree of translational freedom and to the orientation of that object or portion of the object in at least one degree of rotational freedom (up to six total degrees of freedom).

Although some of the examples described herein refer to surgical procedures or instruments, or medical procedures and medical instruments, the techniques disclosed optionally apply to non-medical procedures and non-medical instruments. For example, the instruments, systems, and methods described herein may be used for non-medical purposes including industrial uses, general robotic uses, and sensing or manipulating non-tissue work pieces. Other example applications involve cosmetic improvements, imaging of human or animal anatomy, gathering data from human or animal anatomy, and training medical or non-medical personnel. Additional example applications include use for procedures on tissue removed from human or animal anatomies (without return to a human or animal anatomy) and performing procedures on human or animal cadavers. Further, these techniques can also be used for surgical and nonsurgical medical treatment or diagnosis procedures.

A computer is a machine that follows programmed instructions to perform mathematical or logical functions on input information to produce processed output information. A computer includes a logic unit that performs the mathematical or logical functions, and memory that stores the programmed instructions, the input information, and the output information. The term “computer” and similar terms, such as “processor” or “controller” or “control system,” are analogous.

While certain exemplary embodiments of the invention have been described and shown in the accompanying drawings, it is to be understood that such embodiments are merely illustrative of and not restrictive on the broad invention, and that the embodiments of the invention not be limited to the specific constructions and arrangements shown and described, since various other modifications may occur to those ordinarily skilled in the art.

1. A system comprising:
a processor; and
a memory having computer readable instructions stored thereon, the computer readable instructions, when executed by the processor, cause the system to:
receive three-dimensional primary image data from an imaging system with a field of view;
generate a surface representation for a tissue surface in the field of view;
identify an area in the field of view with a fiducial marker;
associate the fiducial marker with the surface representation; and
move the surface representation and the fiducial marker in response to motion of the tissue surface in the field of view.

2. The system of claim 1, wherein the three-dimensional primary image data is of a field of view illuminated with by visible spectrum light.

3. The system of claim 1, wherein the surface representation includes a mesh model, a point cloud model, or a continuous surface model.

4. (canceled)

5. (canceled)

6. The system of claim 1, wherein the surface representation is generated by a simultaneous localization and mapping rendering technique.

7. The system of claim 1, wherein the surface representation is generated by a neural radiance field rendering technique.

8. The system of claim 1, wherein identifying an area in the field of view with a fiducial marker includes receiving augmented image data of the field of view from a secondary imaging modality.

9. The system of claim 8, wherein the augmented image data is received from a co-registered secondary imaging modality.

10. The system of claim 8, wherein the secondary imaging modality includes a fluorescence imaging modality.

11. The system of claim 8, wherein the secondary imaging modality is at least one of a hyperspectral imaging modality, a laser speckle contrast imaging modality, an oxygenated/deoxygenated hemoglobin concentration imaging modality, or a Raman spectroscopy imaging modality.

12. The system of claim 8, wherein identifying an area includes receiving a user indication of a structure visible in the augmented image data.

13. The system of claim 8, wherein identifying an area includes receiving an identification of a structure visible in the augmented image data from a trained recognition system.

14. The system of claim 8, wherein the fiducial marker is visible when the augmented image data is suppressed or dissipated.

15. The system of claim 1, wherein the computer readable instructions, when executed by the processor, further cause the system to display the fiducial marker associated with the identified area.

16. The system of claim 1, wherein fiducial marker is a three dimensional fiducial marker.

17. The system of claim 1, wherein the fiducial marker corresponds to a set of sampled points projected to the surface representation.

18. The system of claim 1, wherein moving the surface representation includes tracking displacement and deformation of the tissue surface in the field of view using a surface rendering technique.

19. The system of claim 1, wherein moving the fiducial marker includes tracking displacement or deformation of the fiducial marker associated with the tissue surface or associated with motion of the imaging system.

20. (canceled)

21. The system of claim 1, further comprising modifying the surface representation by a simultaneous localization and mapping rendering technique in response to a displacement or deformation of the tissue surface or in response to a motion of the imaging system.

22. (canceled)

23. The system of claim 1, wherein a first portion of the fiducial marker moves relative to a second portion of the fiducial marker, in response to the motion of the tissue surface.

24. The system of claim 1, wherein the computer readable instructions, when executed by the processor, further cause the system to display the moved fiducial marker as an overlay on the three-dimensional primary image data.

25-49. (canceled)
